(19) United States
(12) Patent Application Publication
(10) Pub. No.: US 2023/0112437 A1
(43) Pub. Date: Apr. 13, 2023

(54) System and Method for Secure Data Exchange and Management

(71) Applicant: The Trustees of Indiana University, Bloomington, IN (US)

(72) Inventor: Qingxue Zhang, Carmel, IN (US)

(21) Appl. No.: 17/496,268

(22) Filed: Oct. 7, 2021

(51) Int. Cl. G16H 10/60; G06F 21/62; G16H 40/20; G16H 40/67

(52) U.S. Cl. CPC G16H 10/60; G06F 21/6245; G16H 40/20; G16H 40/67

(57) ABSTRACT

Method and system for acquiring and handling health data from patients using patient and doctor devices and a central system. The patient device gets real time sensor readings and transmit the readings to the central system. The patient device can visualize the readings. The central system stores the readings; and enables doctor devices to access the readings. The central system determines the patient set associated with the doctor, and allows the doctor to access and visualize the readings of any patients in their patient set. Visualization can include creating time series graphs of the readings. The doctor and patient can communicate through the central system.

This invention was made with government support under 2047849 awarded by National Science Foundation. The Government has certain rights in the invention.

### FIELD OF THE DISCLOSURE

The present disclosure relates to the exchange and management of data, and more specifically relates to the exchange and management of healthcare data.

### BACKGROUND

The world is becoming consumed by mass production and consumption of data. The amount of data generated by the world continues to increase exponentially each year and shows no signs of stopping. It is predicted that the annual size of the global data-sphere will increase to 175 ZB by 2025. We rely more heavily on this data to make informed decisions. Due to these increases, new challenges arise to manage the massive amount of data, sometimes called “big data”. As a result, systems from different industries must now adapt to deal with these challenges, and new technologies to manage this data driven world must be developed and continually improved. Big data has multiple characteristics, including volume, velocity, and variety. It is pressing to develop innovative and effective systems to manage this data.

The era of big data is advancing in many areas including healthcare, which requires smart and secure transmission and management of data, including both patient information and patient records. There are some previously reported healthcare data systems for data capturing and management. Some systems are directed to data capturing using wearable sensors, and have not developed the interactive data management aspects. However, capturing and storing big data in network accessible data stores, sometimes referred to as “the cloud”, is important for medical decision support. Other systems have limited data sharing features and have not fully implemented important data management features, such as secure doctor and patient management, interactive chatting, and visualization functions. Therefore, healthcare is still lacking a system that can effectively, securely, and in a smart way, manage big data.

Mobile edge devices, for example smart phones, tablets, etc., are a natural source of big data and are becoming ubiquitous in our daily lives. Edge devices offer a variety of sensors that are built-in, and edge devices can receive or retrieve data from other sensors, for example home medical devices, etc. This makes edge devices a valuable source of data that can be used for healthcare monitoring. Since this data is coming directly from personal devices, the generated data is sensitive and must be handled in a smart and secure way. In addition to generating data, it is also desirable to interact with the big data. Therefore, it is critical to create edge systems that enable users to access their data and ensure that these applications are smart and secure.

There are previously reported mobile systems for healthcare, but with limitations. Some mobile applications can visualize the real-time data for the users, but to further boost the potential of big data, it is important to stream this data to the cloud in a smart and secure way. So far, it is still pressing to develop a system that can effectively secure the data transmission. Further, it is important to provide an interactive interface that facilitates communication between the edge user (e.g., a patient) and the cloud user (e.g., a doctor).

It would be desirable to have a system and method that can effectively and securely collect and manage healthcare data to enhance healthcare systems, better monitor patients, give doctors access to patient data, and provide effective communication between patients and doctors.

### SUMMARY

A method and system are disclosed for acquiring and handling health data from multiple patients. The method includes enabling a central system to communicate with edge devices, where each of the edge devices is associated with a particular patient. For each particular edge device the method includes: configuring the edge device to receive continuous and real time readings from one or more sensors that monitor physical parameters of the patient associated with the edge device; enabling the patient to select a sensor set for transmitting readings to the central system. The edge device can include a transmit activation selection, such that, when the transmit activation is activated, the edge device transmits the continuous and real time readings received from the selected sensor set to the central system; and when the transmit activation selection is deactivated, the edge device does not transmit the continuous and real time readings to the central system. The method also includes enabling visualization of the continuous and real time readings received from the selected sensor set with the edge device. For the central system, the method includes receiving the continuous and real time readings from the edge devices on which the transmit activation selection is activated; storing the received readings; and enabling doctor devices to access the data. Each doctor device is associated with a particular doctor, and each doctor is associated with a patient set. The central system accepts requests from a doctor device; determines the doctor associated with the doctor device; and determines the patient set associated with the doctor. The central system allows the doctor to access and visualize the continuous and real time readings of any of the patients in the patient set associated with the doctor; and does not allow the doctor to access or visualize the continuous and real time readings of any of the patients not in the patient set associated with the doctor. Visualization of the continuous and real time readings can include creating a time series graph of at least a portion of the continuous and real time readings.

The edge devices can include patient smartphones, where each patient smartphone is associated with one of the patients. A portion of the one or more sensors can be built into the patient smartphone. The one or more sensors can include a medical monitoring device that monitors a physical parameter of the patient.

The method can also include enabling real time communication between a doctor and a patient in the patient set associated with the doctor through the doctor device associated with the doctor and the edge device associated with the patient. The communication between the patient and the doctor can be enabled with electronic messages sent between the patient smartphone and the doctor device; and all the electronic messages can be stored by the central system.

Access to the continuous and real time readings can be limited to the patient through their smartphone and an authorized doctor associated with the particular patient through the central system.

Each of the smartphones can be configured to start transmission of the continuous and real time readings from the selected sensor set to the central system upon activation of the transmit activation selection, and to cease transmission of the continuous and real time readings from the selected sensor set to the central system upon deactivation of the transmit activation selection.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 illustrates a top level overview of an exemplary embodiment for a healthcare data management system;

FIG. 2 illustrates an overview of an exemplary patient edge system;

FIG. 3 illustrates an exemplary flow for communication between a doctor and a patient through the patient chat module;

FIG. 4 illustrates an exemplary records interface screen for the patient records module;

FIG. 5 illustrates an overview of an exemplary physician system;

FIG. 6 illustrates an exemplary protocol for signing up and signing in on a physician system;

FIG. 7 illustrates an exemplary patient manage interface in the patient manage module;

FIG. 8 illustrates an exemplary edit patient window in a patient manage interface of the patient manage module;

FIG. 9 illustrates an exemplary add patient record window that can be brought up in the records manage module;

FIG. 10 illustrates and exemplary protocol for reading and editing patient records by a doctor using the physician system;

FIG. 11 illustrates a protocol for requesting historical patient sensor data for visualization through the dashboard module;

FIG. 12 illustrates an exemplary visualization window with time-series graphs of captured sensor data for a patient;

FIG. 13 illustrates an exemplary visualization screen on a patient edge device with time-series graphs showing historical data for the patient; and

FIG. 14 illustrates co-visualization of captured sensor data by a patient and a doctor where the left-side illustrates a patient window in the patient visualize module on their patient edge device, and the right-side illustrates a doctor window in the doctor dashboard module on their healthcare interface device.

Corresponding reference numerals are used to indicate corresponding parts throughout the several views.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

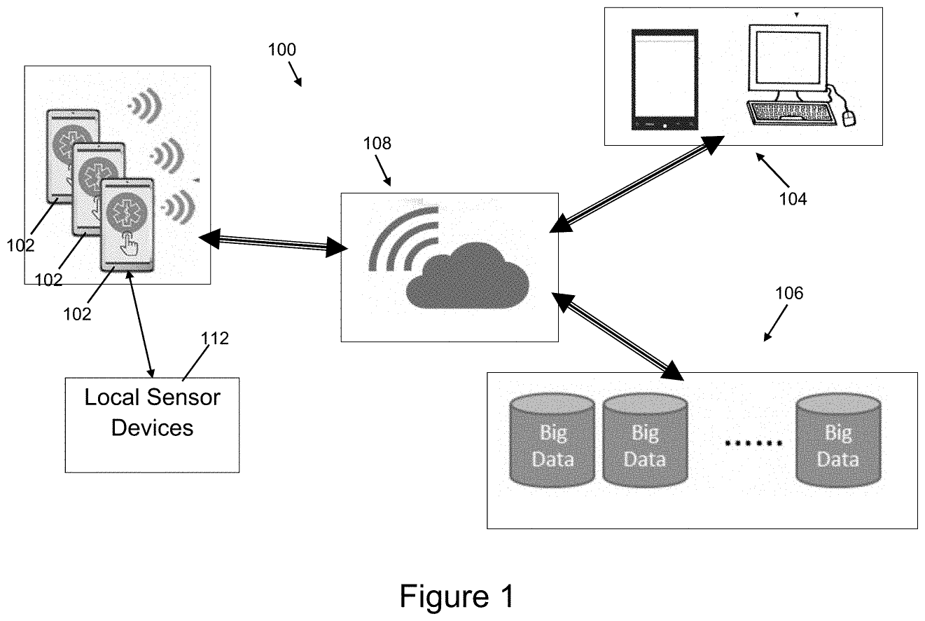

The role of mobile and remote health monitoring is becoming more and more popular, and the demand for ubiquitous healthcare is ever increasing. Patient health is very important and technology has enabled doctors to more successfully monitor, analyze and improve the health of their patients. The healthcare data management system disclosed herein helps to better monitor patients and give doctors critical insights into their patients' health. FIG. 1 illustrates a top level overview of an exemplary embodiment for a healthcare data management system 100 that includes patient edge devices 102, doctor or healthcare interface devices 104 and big data storage 106 all interconnected by a central system 108. The data management system presented herein is also broadly adaptable to benefit and advance other smart home and industry big data applications.

The healthcare data management system 100 can provide several highly desired features. The healthcare data management system 100 can enable long-term, 24-hour, continuous capturing and management of patient data. The healthcare data management system 100 can provide connectability of diverse health monitors to provide improved access to various patient data. The healthcare data management system 100 can provide secure data management and retention, with user sign up and log in security functions, as well as data transmission protection. The healthcare data management system 100 can provide big data visualization on patient edge devices. The healthcare data management system 100 can provide direct connection between doctor and patient via chat for patient-doctor interaction. The healthcare data management system 100 can provide an interactive cloud interface for the doctor, which allows the doctor to easily access patient data, and communicate with patients. The healthcare data management system 100 can enable doctors to conveniently manage both patient information and patient records. The healthcare data management system 100 can support both real-time and historical visualization and analysis of the patient data. The healthcare data management system 100 can provide comprehensive patient management and big data communication for secure system interaction.

A major way to monitor, analyze and improve a patient's health is to make informed decisions based on collected patient data. The source of this data can be the patient edge devices 102 that many patients already have, for example smartphones, tablets, computers, home medical devices, etc. Each patient edge devices 102 is associated with a patient signed up in the healthcare data management system 100. These edge devices 102 can also communicate with one or more local sensors 112, for example home medical devices, patient monitoring devices or other sensors, and transmit data from those local sensors 112 for review and analysis through the data management system 100.

A way to harness the power of the edge devices 102 is to connect them to the central system 108 via a patient system 200. Connecting patient edge devices 102 to the central system 108 creates a “mobile edge” around the data management system 100. The patient system 200 on the patient edge devices 102 plays two important roles. First, the patient system 200 is the source of raw data that flows into the central system 108. Second, the patient system 200 is what connects patients to their health information and their doctors.

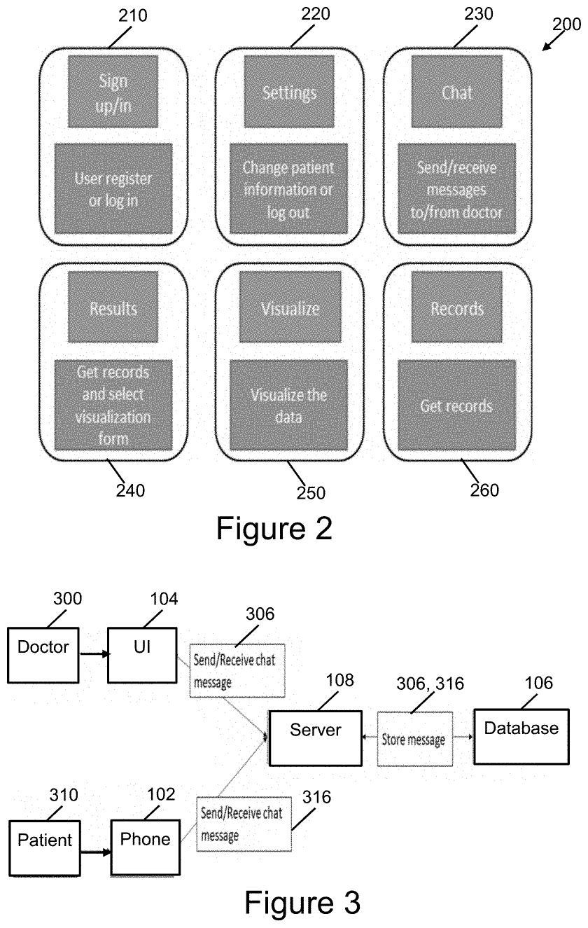

FIG. 2 illustrates an overview of an exemplary patient edge system 200 that can run on the patient edge devices 102 to obtain essential patient data and send it to the central system 108. The patient system 200 provides a smart and secure interface for patients to get full access to their health data, to receive real-time views of their captured sensor data, and to communicate with their doctors. The patient system 200 can include a patient sign up/in module 210, a patient settings module 220, a patient chat module 230, a patient results module 240, a patient visualize module 250 and a patient records module 260.

Data security is an important aspect of the healthcare data management system 100. In order to keep patient data safe, the patient sign up/in module 210 ensures that the patient's data is secured behind a set of unique security credentials. Every patient must have login credentials to access their data, so that no unauthorized person can access their data. The patient sign up/in module 210 securely logs in, signs up, and signs out users, as well as connects users to the vast amounts of data they generate. The patient system 200 allows patients to retrieve and view their data and records, as well as communicate with their physicians. Data is not shared and there is no way to access another patient's data without accessing their account or being their doctor. The patient system 200 can only be used by a user to access the data management system 100 after the user has entered currently recognized security credentials, for example username and password, in the patient sign up/in module 210. Personal patient data and information should not be accessible by unauthorized users. In order to combat security breaches, the patient edge system 200 can use tested security and authentication features in the patient sign up/in module 210. For example, Amazon Web Services Cognito can be used in the patient sign up/in module 210 to safely store and verify user credentials. The patient sign up/in module 210 provides authentication, authorization, and user management for the patient edge system 200. Every patient has a unique identifier that allows them to access their data. This identifier is used throughout the healthcare data management system 100 in order to retrieve data for that specific patient only. Every patient must have login credentials to access their data, so that no unauthorized person can access their data. Additionally, each patient that signs up with the patient edge system 200 has their credentials securely saved through the patient sign up/in module 210

The patient settings module 220 enables patients to view and update their information, for example, name, height, weight, account login, etc. Patients can change their information as needed as well as sign out of the mobile application using the patient settings module 220.

The patient chat module 230 enables patients to directly send messages to and receive messages from their doctors so they can be quickly informed and updated with any new information their doctor has learned. Doctor-patient communication is necessary for successfully improving and monitoring a patient's healthcare. This gives patients direct access to their doctors. All messages sent through the patient chat module 230 are stored in the data storage 106 so that no messages are ever lost, and that all conversation history can be viewed. Through the patient chat module 230, doctors can quickly update their patients on any new feedback with their health, and patients can quickly receive replies from their doctor when they have medical questions.

FIG. 3 illustrates an exemplary flow for communication between a doctor 300 and a patient 310 through the patient chat module 230. The doctor 300 uses their healthcare interface device 104 to send/receive a message 306 to/from the patient 310. The patient 310 uses the patient chat module 230 on their patient edge device 102 to create a message 316 for the doctor 300, and sends the message 316. The messages 306, 316 are routed through the central system 108. The message 306 from the doctor 300 goes from the healthcare interface device 104 through the central system 108 and is routed to the patient edge device 102 associated with the recipient patient 310. The message 316 from the patient 310 goes from the patient edge device 102 through the central system 108 and is routed to the healthcare interface device 104 associated with the recipient doctor 300. The healthcare data management system 100 also stores the messages 306, 316 in the data storage 106.

The patient results module 240 enables patients to view their associated health records and start visualization setup for data viewing. The patient results module 240 enables the patient to access real-time or past sensor data captured by sensors on or through their patient edge device 102. From the patient results module 240, the patient can select which record data they want to view, and which sensor data they want to view, and on which date(s). Once the patient submits the request though the patient results module 240, the central system 108 processes the patient record request and retrieves the requested data from the data storage 106. The requested data is returned to the patient edge system 200 on the patient edge device 102 and a time-series graph is displayed through the patient visualize module 250 showing the requested data that was captured and retrieved.

The patient visualize module 250 enables patients to visualize their current real-time patient data as well as historical stored patient data. Real-time analysis is conducted by retrieving sensor data from the patient's edge device 102 and sending it to the central system 108. Upon starting up the real-time analysis feature, a direct connection is made to the central system 108 and data is captured from the local sensors 112 and/or sensors built into the patient's edge device 102 and sent to the central system 108. This data capture feature is only activated when the patient turns on the sensor in the patient visualize module 250. The patient always has the option to turn off one or more sensors on their edge device 102 to stop further data capture. Not all sensors available to the patient's edge device 102 will be captured. Only the necessary sensors accessible by the patient's edge device 102 that are listed by the doctor are used for data capture. The patient visualize module 250 can show time-series graphs of the captured data from each sensor that is being captured.

The patient records module 260 enables patient review of their health records. Each patient has a doctor that they work with. In order to maintain and improve the health of their patients, the doctors will create health records to track a specific aspects of their patient's health. These health records and their associated analysis are made available to the patient via the patient records module 260 which can list the health records associated with the patient.

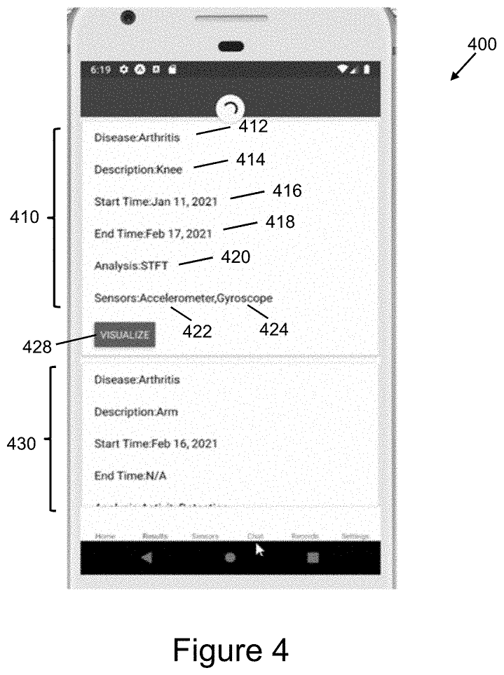

An exemplary records interface screen 400 for the patient records module 260 is illustrated in FIG. 4. The records interface screen 400 shows a first patient record 410 and the top of a second patient record 430. The first record 410 shows a disease name 412, a disease description 414, a record start time 416, a record end time 418, an analysis identifier 420, and sensor identifiers 422, 424. Each sensor identifiers 422, 424 identifies a sensor on the patient's edge device 102 or a local sensor 112 that communicates with the patient's edge device 102 and provides readings that are captured to monitor the patients disease 412. This can help the patients understand what is being analyzed so that there is more transparency between the doctor and patient. The patient can select the visualize selection 428 associated with one of the patient records 410, 430 to get further information about the selected patient record. When the patient selects a particular patient record in the patient records module 260, they are taken to the patient results module 240.

There are two main types of visualization that are available on the patient edge device 102: real-time visualization and historical visualization. Real-time visualization is conducted by retrieving sensor data from/through the patient edge device 102 and sending it to the central system 108. Real-time visualization is activated when the patient turns on a sensor in the patient visualize module 250. The patient always has the option to turn off sensor data capture from/through their patient edge device 102. Not all sensors available on the patient edge device 102 will be captured, but only the sensors that are listed by the doctor in one or more patient records 410, 430.

Historical visualization enables a patient to view their past captured data. This historical visualization can be viewed through the patient results module 240. The patient results module 240 lists health records associated with the patient and enables the patient to view the activity data associated with a particular patient record. The patient is given the option of what sensor data they want to view and from which date. Once the patient submits a request to view the data, the data is retrieved from the data storage 106 and visualized in a time-series graph that the patient can cycle through and examine.

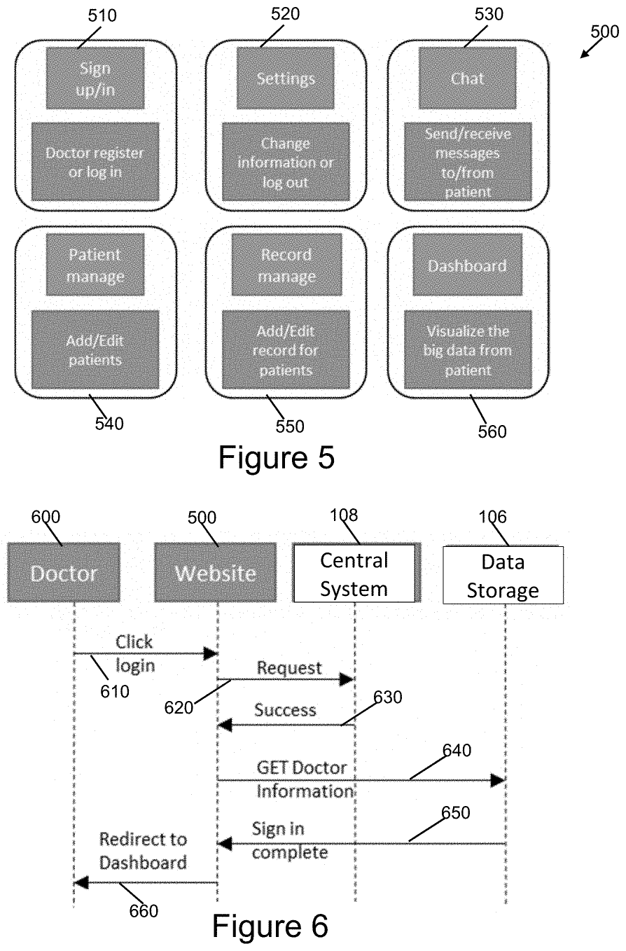

A physician system 500 enables doctors, through their healthcare interface devices 104 to effectively manage both patients and their records. The physician system 500 enables doctors to add and edit patient and record information, to communicate with their patients, and to manage and review both real-time and historical patient data. FIG. 5 illustrates an overview of an exemplary physician system 500 that physicians access through their healthcare interface devices 104. The physician system 500 can include a doctor sign up/in module 510, a doctor settings module 520, a doctor chat module 530, a patient manage module 540, a records manage module 550 and a dashboard module 560.

Security is a critical component of healthcare data systems. The healthcare data management system 100 provides a mobile-cloud collaboration system to provide secure data access. In order to keep health data safe, the doctor sign up/in module 510 verifies user credentials before access to the healthcare data management system 100 is granted. Login credentials are required for every doctor to access their data. This is done to ensure no unauthorized user has access to the data. Doctors have a unique identification number and login credentials that are used to help retrieve their data. Data is not shared with anyone, and access to data associated with a particular doctor is only possible after verification of the account credentials of that doctor in the doctor sign up/in module 510.

Each doctor that signs up with the healthcare data management system 100 has their credentials securely stored by the system. An exemplary protocol for signing up and signing in is illustrated in FIG. 6. A physician 600 interacts with healthcare data management system 100 through the physician system 500. When initially signing up, the doctor 600 enters doctor identification information (e.g. name, email, hospital, etc.). After successfully signing up, the doctor 600 only has to enter their login information. Once this data is filled out in the doctor sign up/in module 510 on the physician system 500, a login or enter selection 610 is selected. When the login selection 610 is made, the doctor sign up/in module 510 sends a request 620 to the central system 108 where the account is created with the credentials the doctor entered. If account creation is successful, then the central system 108 sends a verification code 630 to the doctor's email. The doctor must enter the verification code 630 into the doctor sign up/in module 510 to verify their email. When the verification code 630 is entered into the doctor sign up/in module 510, the healthcare data management system 100 stores the doctor information 640 on the server 106 and the signup is complete 650, at which point the doctor is redirected at step 660 to the doctor dashboard module 560.

Upon signing in via the doctor sign up/in module 510, a sign-in request is made to the central system 108. If this request returns that the doctor credentials are valid, then an additional request is made to retrieve that valid doctor's information. Retrieving this valid doctor's information furthers security of the system because the information is used in requests so that only valid doctors can make valid requests.

The doctor settings module 520 enables doctors to view and update their information. Doctors can change their information (e.g., name, specialty, account login, etc.) as well as sign out of the physician system 500.

The doctor chat module 530 enables the doctor to directly communicate with their patients. Doctors can use the doctor chat module 530 to quickly notify their patients of new information regarding their health, and to receive messages sent by their patients from the patient chat module 230. Likewise, patients can instantly receive replies through the patient chat module 230 that were sent from their doctor through the doctor chat module 530 about any questions or concerns they have regarding their health. Doctors and patients use the chat feature by typing out a message and pressing the submit button. All messages that are sent go through the healthcare data server 108 and are saved in the data storage 106. Messages are available for viewing whenever the conversation is opened up. When the conversation is rendered, the data associated with each message (e.g. sender, date, content) are displayed. If the sender was the doctor then the message can be colored in one color, and If the sender was the patient then the message can be colored in a different color.

The patient manage module 540 enables doctors to add patients to their patient list, and to create health records for their patients. Doctors can create health records to help organize, track, and improve their patients' health. Doctors typically have multiple patients that are under their care. The patient manage module 540 can provide a list of all of the current patients that the doctor is treating and their associated data (e.g. patient id, height, weight, etc.). The patient manage module 540 enables the doctor to add new patients to their care, and to remove existing patients from their care.

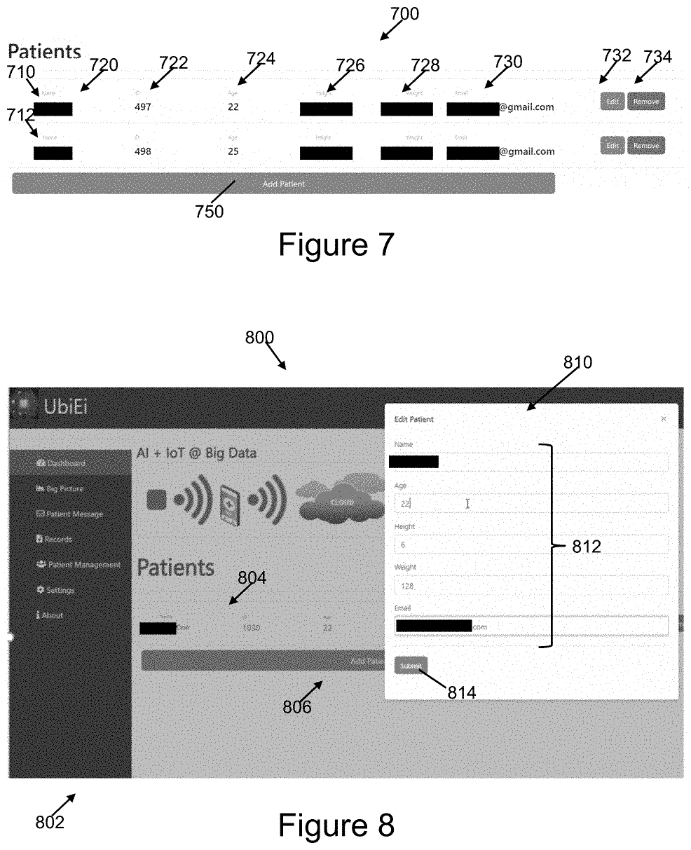

FIG. 7 illustrates an exemplary patient manage interface 700 in the patient manage module 540 showing two patient entries 710, 712 and an add patient selection 750. Each patient entry 710, 712 includes fields for patient name 720, patient ID 722, patient age 724, patient height 726, patient weight 728, and patient email 730. Each patient entry 710, 712 also includes and edit selection 732 and a remove selection 734. The doctor can select the add patient selection 750 to add more patients. The doctor can select the edit selection 732 to change the patient information for the selected patient entry. The doctor can select the remove selection 734 to delete the patient entry from their records. With his patient management page, the doctor can easily go through and/or update the patient information if needed.

FIG. 8 illustrates an exemplary edit patient window 810 in a patient manage interface 800 of the patient manage module 540. The patient manage interface 800 includes a navigation bar 802 with links to each of the modules 510-560 of the physician system 500, includes a list of patients 804 under the doctor's care, and includes an add patient selection 806. Selecting the add patient selection 706 brings up the edit patient window 810. The edit patient window 810 includes patient data fields 812 for the doctor to enter patient information (e.g., name, age, height, weight, email, etc.), and a submit selection 814. When the patient data fields 812 are complete, the submit selection 814 can be selected to create a new patient.

The records manage module 550 enables doctors to create and view the health records for their patients and their associated analysis. The records manage module 550 provides access to all the health records associated with each patient. These health records are used to help monitor patients so that doctors can maintain and improve patient health. Upon opening the records manage module 550, a request is made to the server 106 to retrieve all records of patients associated with the signed in doctor. Doctors can add new records for their patients. Adding a record will bring up a separate add patient record window.

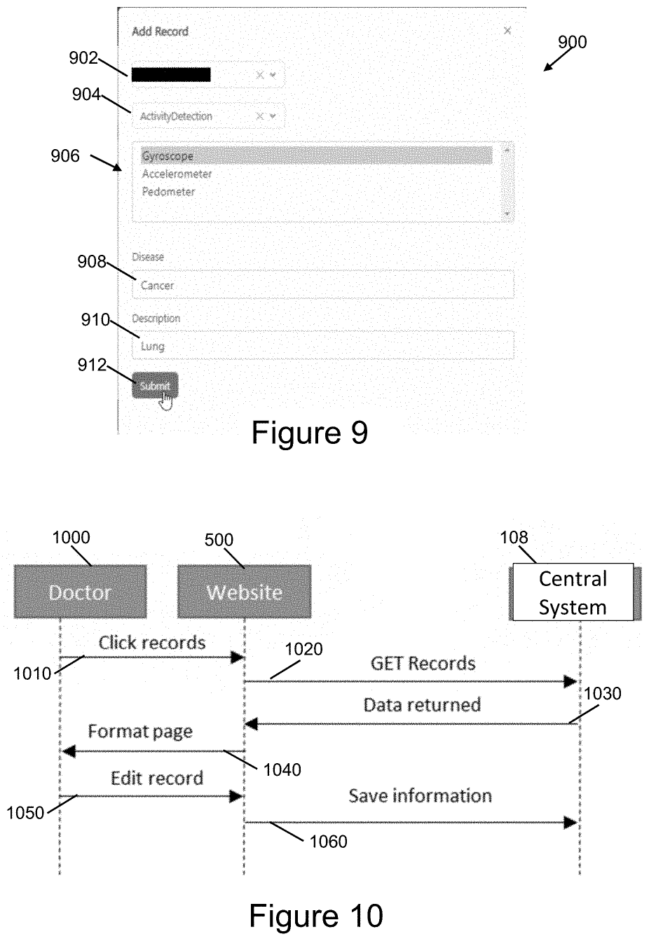

FIG. 9 illustrates an exemplary add patient record window 900 that can be brought up in the records manage module 550. The add patient record window 900 can include a patient name or identifier field 902, an analysis type field 904, a sensor capture list 906, a disease type field 908, a record description field 910, and a submit selection 912. The add patient record window 900 can also include other fields, for example start and end time fields. The sensor capture list 906 includes one or more sensors available to the healthcare data management system 100 through the patient edge device 102 associated with the patient. The doctor can select one or more of the sensors in the sensor capture list 906 for data collection in monitoring and treatment of the patient. The doctor will need to fill out the different fields in the add patient record window 900 and then select the submit selection 912 to create a new patient record.

Once a doctor adds a record, that record will show that the doctor can edit or remove the record using the edit selection 732 and a remove selection 734 options shown in FIG. 7. If the doctor edits a record, an edit patient record window, similar to the add patient record window 900, will be displayed. The edit patient record window will display all of the current values for each field of the record. The doctor can then modify the entries in the fields of the patient record.

FIG. 10 illustrates and exemplary protocol for reading and editing patient records by a doctor 1000 using the physician system 500. The doctor 1000 selects the records manage module 550 at step 1010, which prompts the physician system 500 to request all the patient records of the doctor 1000 from the central system 108 at step 1020. At step 1030 the patient records are returned to the physician system 500, and at step 1040 the records manage module 550 formats a records interface to display the returned patient healthy records. At step 1050 the doctor 1000 can edit one of the patient records using the records manage module 550, and at step 1060 the physician system 500 sends the edited record data to the central system 108 to be saved in the data storage 106.

The dashboard module 560 enables doctors to view the patient data. Real-time visualization can be used to view the real-time data for one of their patients coming from the patient's edge device 102. The dashboard module 560 also enables doctors to view historical sensor data for their patients.

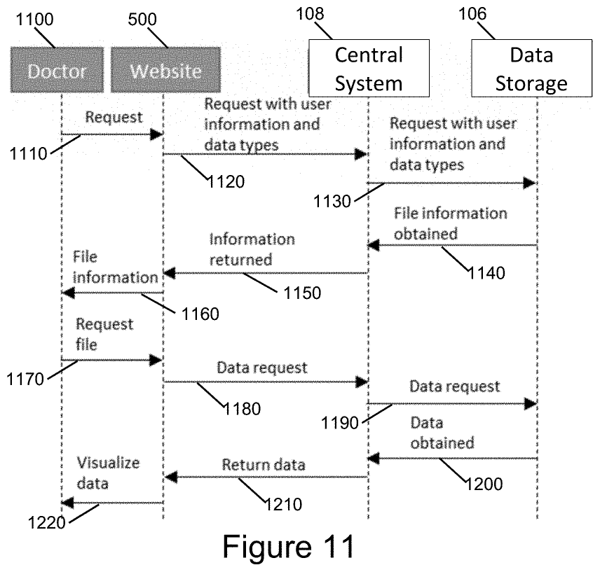

The dashboard module 560 can include a historical visualization selection that enables a doctor access and display previously captured and stored sensor data. Upon pressing the historical visualization selection, a historical visualization request form can be shown where a doctor selects the desired patient, the patient's record, one of the sensors that is on the selected patient record, and the date the sensor data was sent to the healthcare data management system 100. FIG. 11 illustrates a protocol for this process. At step 1110 a doctor 1100 selects the historical visualization selection in the dashboard module 560 of the physician system 500 on their doctor interface device 104. At step 1120, the doctor 1100 fills out part of the historical visualization request form with the desired patient, the patient's record and the desired sensors, and the doctor 1100 submits this information to the central system 108. At step 1130, the central system 108 forwards this information to the data storage 106. At step 1140, the data storage 106 determines the data stored for the desired patient, patient record and the sensors and sends this file information to the central system 108. At step 1150, the central system 108 forwards the file information to the physician system 500 on the doctor interface device 104. At step 1160, the dashboard module 560 populates the historical visualization request form with the available dates for the requested sensor data. At step 1170, the doctor 1100 makes the remaining selections on the historical visualization request form in the dashboard module 560. At step 1180, the doctor 1100 submits the completed data request to the central system 108. At step 1190, the central system 108 forwards the data request to the data storage 106. At step 1200, the data storage 106 retrieves the requested data and sends it to the central system 108. At step 1210, the central system 108 forwards the requested data to the physician system 500 on the doctor interface device 104. At step 1220, the requested historical sensor data is displayed as a time-series graph in the dashboard module 560.

The time-series graphs of the sensor data can have several controls that enable users to better review the data, for example zoom-in, zoom-out and playback controls. One challenging task associated with visualization is viewing large amounts of data at once. Since a large amount of data can be kept in cloud storage 106, retrieval and visualization can be difficult. Some reasons for this difficulty include timeouts and slow response time while retrieving large amounts of data. To address these problems, data can be retrieved in smaller amounts and for shorter time intervals. Since all of the data is not usually viewable at once, the doctor can use playback controls (backward, forward, play, stop) that allow them to scroll through the retrieved data. If the doctor scrolls past the end of the retrieved data already stored locally, then a request can be made to retrieve additional data. Each time a request is made, the data is appended to the locally stored data accessible to the physician system 500. These zoom and playback controls enable the doctor to closely analyze all or any desired portion of a patient's data without being hindered by the size of the data.

Experiments have been conducted to test the functions of patient edge system 200, the physician system 500, the central system 108 and their interactions through the healthcare data management system 100. Test patients were signed up and established test accounts using patient edge devices 102. The test patients logged into the healthcare data management system 100 on their patient edge device 102. Through their test accounts, the test patients could view and update their patient information; could visualize real time sensor data; could visualize historical sensor data; and could chat with their doctor. The physician system 500 was also tested by setting up test accounts for doctors using the respective signup pages. Afterwards, different pages with various functions were tested.

Upon startup of the patient edge system 200 on their patient edge device 102, the patient is prompted to login or signup. The process of authorizing patients during logging in or signing up is managed by the patient sign up/in module 210. Once a patient is fully signed up with the system, a doctor can connect with the patient through the patient manage module 540, and start helping them analyze their health

The physician system 500 includes different interface pages for the various modules 510-560 that enable the doctor to fully manage and monitor their patients. Doctors can navigate the physician system 500 using the navigation bar 802 on the left side of FIG. 8. The navigation bar 802 can be available on all pages of the physician system 500. Upon selecting a different tab within the navigation bar 802, the doctor is taken to the corresponding module 510-560 of the physician system 500.

For security testing, doctors were signed up with the doctor sign up/in module 510 of the physician system 500. When a doctor attempts to sign up, they must fill out required identifying information, for example name, email, phone number(s), hospital, medical specialty and login credentials. When the required information is completed, it is sent to and verified by the central system 108. After verification is completed, the doctor's information can be saved to the data storage 108 and the doctor is given a unique identifier. Once this is complete, the doctor can be redirected to the dashboard module 560. When a doctor that is already in the physician system 500 attempts to sign in, they can just enter their username or email, and a password.

To test the chat features of the patient chat module 230 and the doctor chat module 530, messages were sent back and forth between doctor and patient. Since doctors have multiple patients, if they are not already conversing with the desired patient in the doctor chat module 530, the doctor must first select the desired patient they wish to converse with. Correct messages were displayed in the patient and doctor chat modules 230, 530, and the messages were saved on the healthcare data management system 100. In order to communicate through the chat feature, a sender can simply type a message into a message box and select a submit selection. The message can then be saved and made available for viewing by the recipient and sender. Each message has sender, date, and content information associated with it. This enables direct connection between a doctor and their patient, so that patients no longer feel disconnected from their doctors and unsure about their health. Doctors can also quickly inform patients of new information on their health.

The visualization features of the patient edge system 200 and the physician system 500 for real-time data or historical data were also tested. In the dashboard module 560, the doctor can select a desired patient for review, and the physician system 500 can retrieve the patient sensor data and present as a time-series graph.

For the real-time case, a test patient account was setup and was used to send real-time test data. When data is sent from the patient edge device 102, it is saved to the cloud storage 106 in real-time. A doctor account was used to visualize the real-time data sent from the patient edge device 102. Before real-time data can be viewed, the patient must be actively sending data. The central system 108 keeps track of whether a patient is currently active or not. If a patient is not active then the doctor will not be able to view real-time data. If the patient is active then a real-time visualization request can be sent. The system is setup so that only authenticated users can view the data. Once this occurs, the system determines the necessary information to view the real-time data. The time-series graph visualizes the data within a certain window length. If the data exceeds this window, then the time-series graph is shifted so that the new data can be viewed.

For the historical case, patient sensor data stored in the data storage 106 was used to visualize. The doctor can view data from a specific patient. To view data, the doctor makes selections using the historical visualization form in the dashboard module 560. Once the information is selected, a request is submitted to retrieve the stored patient data from the data storage 106. This request initiates the data retrieval process. The request is processed and the data retrieved. The data can be down-sampled before being sent to the requesting doctor. The dashboard module 560 creates a time-series graph to visualize the retrieved data.

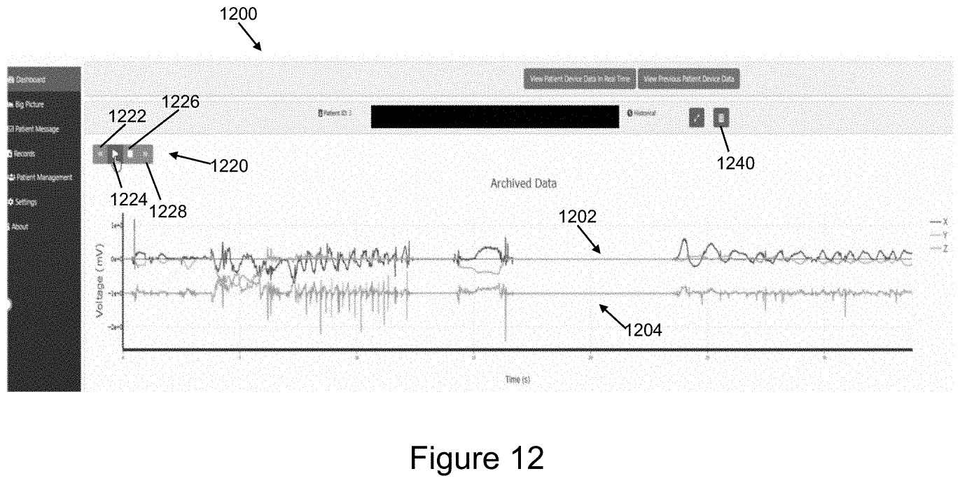

FIG. 12 illustrates an exemplary visualization window 1200 with time-series graphs 1202, 1204 of captured sensor data for a patient. The visualization window 1200 includes playback controls 1220 that enable the doctor to scroll through the data. The playback controls 1220 can include a rewind control 1222 to scroll back, a play control 1224 to scroll forward, a stop control 1226 to stop scrolling, and a fast forward control 1228 to scroll forward at a faster rate. As the doctor scrolls through the data, a set interval of data is retrieved each time additional data is needed. Each time a request is made, the retrieved data is appended to the data that is already stored locally for the time-series graph. While scrolling, the time-series graph will index through the locally saved data and add the newly retrieved data to the visualization. If the time-series graph reaches the end of the retrieved data, then it will make a new request to get the next needed interval of data. Note that the graphs 1202, 1204 only visualize a certain window of data at a time, so if more data is added then the graph 1202, 1204 adjusts by shifting to the left so that the new data is displayed on the right. When the doctor is done viewing the sensor data of the patient, they can use the a delete selection 1240 to remove the visualization data. The visualization and data retrieval functions were tested to ensure the doctor could receive, view, and examine the data.

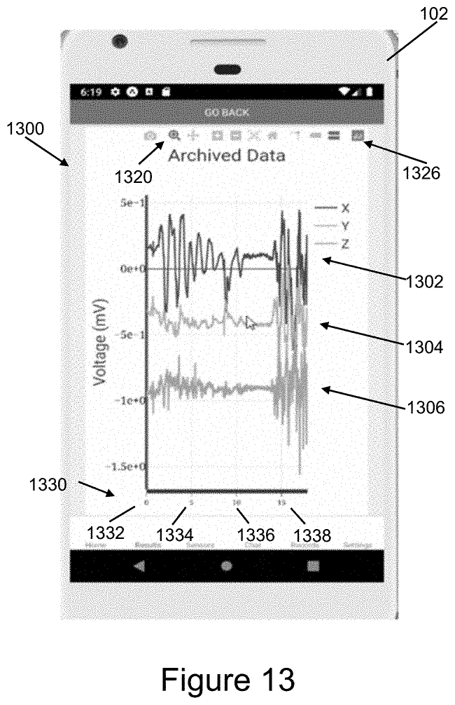

To test visualization of historical data by the patient edge system 200 on a patient edge device 102, the patient results module 240 was selected and a request was submitted to view historical data. FIG. 13 illustrates an exemplary visualization screen 1300 on the patient edge device 102 with time-series graphs 1302, 1304, 1306 showing historical data for the patient, where the data was requested and retrieved from the data storage 106. To test this aspect of the system, the historical data was previously stored for the patient in the data storage 106. The results module 240 of the patient edge system 200 allows patients to view their past history of captured sensor data. When the patient wants to view this data, they must select the record they want to view and the specific sensor data. This data request is created and sent to the data storage 106, which processes the request and returns the data to the patient edge device 102. A time-series graph is created to visualize the returned data. Note that not all of the data is typically viewable at one time. The patient can use a zoom function 1320 to get a closer look at a desired portion of the data. Since there is potentially a large amount of data, it might not be possible to retrieve and visualize all of the data at once. So the system can retrieve the data in set time intervals to reduce the size of the data request. Additional data can be retrieved and appended to the existing data in the time-series graph. The visualization screen 1300 also includes playback controls 1330 that can include a rewind control 1332 to scroll back, a play control 1334 to scroll forward, a stop control 1336 to stop scrolling, and a fast forward control 1338 to scroll forward at a faster rate. When the patient is done looking at their data, they can return back to the main results screen by pressing a back selection 1326. This enables the patient to directly interact with and view their data, which helps the patient be more involved in the health monitoring process.

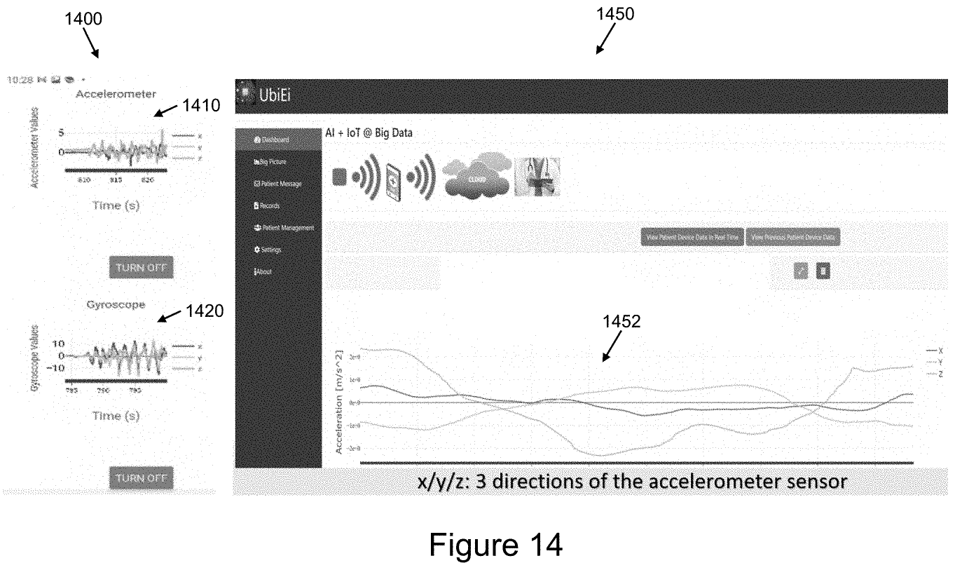

FIG. 14 illustrates co-visualization of captured sensor data by the patient and the doctor. In FIG. 14, the left-side illustrates a patient window 1400 in the patient visualize module 250 on the patient edge device 102, and the right-side illustrates a doctor window 1450 in the doctor dashboard module 560 on their healthcare interface device 104. In order to test the real-time functionality, the patient edge device 102 was actively sending sensor data to the central system 108. The patient window 1400 visualizes the sensor data on a top time-series graph 1410 for an accelerometer sensor, and a bottom time-series graph 1420 for a gyroscope sensor. Different channel axis values are being captured from the associated sensors and graphed. All of the sensor data is sent in real-time from the patient edge device 102 to the central system 108. The patient edge system 200 makes a direct connection to the central system 108 and is able to capture and send the sensor data directly to the central system 108.

The patient's doctor can subscribe to the data that the patient is sending and view it in real-time in the doctor window 1450 on their healthcare interface device 104. Notice that the doctor's graph 1452 and the patient's graph 1410 are very close to in-sync. This means that the doctor gets almost instantaneous updates on the patient's sensor readings for review and analysis. The time-series graphs 1410, 1420, 1452 only show a certain interval of time, so as more data comes in, the time-series graphs will shift so that new data can be shown. Each time-series graph 1410, 1420, 1452 can be equipped with zoom, playback and other controls to enable the patient and doctor to look at only certain portions or increase/decrease the view size. This enables both the doctor and patient to get a close look at the data they desire. This makes it easier for patients to be monitored and involved in the analysis, enables the doctor to do virtually real-time monitoring and analysis of the patient data, and enables better coordination and collaboration between the doctor and the patient.

While the disclosure has been illustrated and described in detail in the drawings and foregoing description, such illustration and description is to be considered as exemplary and not restrictive in character, it being understood that illustrative embodiment(s) have been shown and described and that all changes and modifications that come within the spirit of the disclosure are desired to be protected. It will be noted that alternative embodiments of the present disclosure may not include all of the features described yet still benefit from at least some of the advantages of such features. Those of ordinary skill in the art may readily devise their own implementations that incorporate one or more of the features of the present disclosure and fall within the spirit and scope of the present invention as defined by the appended claims.

We claim:

1. A method for acquiring and handling health data from a plurality of patients, the method comprising:
enabling a central system to communicate with a plurality of edge devices, each of the plurality of edge devices being associated with a particular patient of the plurality of patients;
for each particular edge device of the plurality of edge devices:
configuring the particular edge device to receive continuous and real time readings from one or more sensors configured to monitor one or more physical parameters of the patient associated with the particular edge device;
enabling the patient associated with the particular edge device to select a selected sensor set from the one or more sensors from which the particular edge device is configured to receive continuous and real time readings;
providing a transmit activation selection on the particular edge device;
when the transmit activation selection is activated on the particular edge device, transmitting the continuous and real time readings received from the selected sensor set from the particular edge device to the central system;
when the transmit activation selection is deactivated on the particular edge device, not transmitting the continuous and real time readings from the particular edge device to the central system;
enabling visualization of the continuous and real time readings received from the selected sensor set with the particular edge device;
for the central system;
receiving the continuous and real time readings from the plurality of edge devices on which the transmit activation selection is activated;
storing the received continuous and real time readings by the central system; and
enabling a plurality of doctor devices to access data through the central system, each of the plurality of doctor devices being associated with a particular doctor, and each particular doctor being associated with a patient set of one or more of the plurality of patients;
accepting requests from a particular doctor device of the plurality of doctor devices;
determining the particular doctor associated with the particular doctor device;
determining the patient set associated with the particular doctor;
allowing the particular doctor associated with the particular doctor device to access and visualize the continuous and real time readings of any of the plurality of patients in the patient set associated with the particular doctor; and
not allowing the particular doctor associated with the particular doctor device to access or visualize the continuous and real time readings of any of the plurality of patients not in the patient set associated with the particular doctor.

2. The method of claim 1, wherein visualization of the continuous and real time readings comprises creating a time series graph of at least a portion of the continuous and real time readings.

3. The method of claim 1, further comprising:
enabling real time communication between a particular doctor and a particular patient in the patient set associated with the particular doctor through the doctor device associated with the particular doctor and the edge device associated with the particular patient.

4. The method of claim 1, wherein the plurality of edge devices includes a plurality of patient smartphones, each of the plurality of patient smartphones being associated with one of the plurality of patients.

5. The method of claim 4, wherein a portion of the one or more sensors are built into the plurality of patient smartphones.

6. The method of claim 4, further comprising a medical monitoring device configured to monitor a physical parameter of the patient;
wherein the one or more sensors includes the medical monitoring device.

7. The method of claim 4, wherein access to the continuous and real time readings is limited to the patient through the particular smartphone associated with the particular patient, and an authorized doctor associated with the particular patient through the central system.

8. The method of claim 4, further comprising:
enabling real time communication between a particular doctor and a particular patient in the patient set associated with the particular doctor through the doctor device associated with the particular doctor and the patient smartphone associated with the particular patient.

9. The method of claim 8, wherein communication between the patient and the doctor is enabled with electronic messages sent between the patient smartphone and the doctor device; and all the electronic messages sent between the patient smartphone and the doctor device are stored by the central system.

10. The method of claim 4, wherein each of the plurality of patient smartphones is configured to start transmission of the continuous and real time readings from the selected sensor set to the central system upon activation of the transmit activation selection, and to cease transmission of the continuous and real time readings from the selected sensor set to the central system upon deactivation of the transmit activation selection.

11. A method for acquiring and handling health data from a patient, the method comprising:
associating an edge device with the patient;
configuring the edge device to receive continuous and real time readings from one or more sensors configured to monitor a physical parameter of the patient;
enabling visualization of the continuous and real time readings with the edge device;
receiving the continuous and real time readings from the edge device at the central system when the transmit activation selection is activated;
storing the continuous and real time readings by the central system; and
enabling a plurality of doctor devices to access data through the central system, each particular doctor device of the plurality of doctor devices being associated with a particular doctor, and each particular doctor being associated with a patient set of one or more patients;
when the patient associated with the edge device is in the patient set associated with the particular doctor, allowing access to and visualization of the continuous and real time readings received from the edge device by the particular doctor device associated with the particular doctor.

12. The method of claim 11, wherein visualization of the continuous and real time readings comprises creating a time series graph of at least a portion of the continuous and real time readings.

13. The method of claim 11, further comprising:
when the patient associated with the edge device is in the patient set associated with the particular doctor, enabling real time communication between the edge device and the particular doctor device associated with the particular doctor.

14. The method of claim 11, wherein the edge device is a smartphones associated with the patient.

15. The method of claim 14, wherein at least one of the one or more sensors are built into the smartphone.

16. The method of claim 14, further comprising a medical monitoring device configured to monitor a physical parameter of the patient;
wherein the one or more sensors includes the medical monitoring device.

17. The method of claim 14, wherein access to the continuous and real time readings is limited to the patient through the smartphone associated with the patient and an authorized doctor through the central system, where the patient is in the patient set associated with the authorized doctor.

18. The method of claim 17, further comprising:
enabling real time communication between the authorized doctor and the patient in the patient set associated with the authorized doctor through an authorized doctor device associated with the authorized doctor and the smartphone associated with the patient.

19. The method of claim 14, further comprising:
providing a transmit activation selection on the smartphone;
when the transmit activation selection is activated, transmitting the continuous and real time readings from the smartphone to the central system; and
when the transmit activation selection is deactivated, not transmitting the continuous and real time readings from the smartphone to the central system;

20. The method of claim 19, wherein the smartphone is configured to start transmission of the continuous and real time readings from the selected sensor set to the central system upon activation of the transmit activation selection, and to cease transmission of the continuous and real time readings from the selected sensor set to the central system upon deactivation of the transmit activation selection.
